(19) United States
(12) Patent Application Publication
(10) Pub. No.: US 2005/0251005 A1
(43) Pub. Date: Nov. 10, 2005

(54) Mapping and viewing device for an intervertebral disc

(76) Inventor: Ashish Dhar Diwan, Edgecliff, NSW (AU)

Correspondence Address: HOLLANDER LAW FIRM, P.L.C., SUITE 305, 10300 EATON PLACE, FAIRFAX, VA, 22030, US

(21) Appl. No.: 10/530,152

(22) PCT Filed: Sep. 30, 2003
(86) PCT No.: PCT/AU03/01289

§ 371 Date: Jul. 1, 2005

(30) Foreign Application Priority Data
Oct. 1, 2002 (AU) 2002951762

(51) Int. Cl. A61B 5/05

(52) U.S. Cl. 600/407

(57) ABSTRACT

The invention is system for mapping and imaging the interior of a bodily cavity of a patient. The invention comprising a position indication means (63) variably positionable within the bodily cavity; a position detection means (64) for receiving a signal from the position indication means (63) and a processor means that analyses the signal and provides an output indicative of the location of the position indication means (63) relative to the position detection means (64). The invention also comprises a first imaging means (65) positionable within and for producing a first image of the interior, and at least a second imaging (62) means positionable within and for producing a second image of the interior. The second imaging means (62) is movable relative to the first imaging means (65) and positionable in a location wherein first image depicts the location of the second imaging means (62).

### TECHNICAL FIELD

The present invention relates to a system, device and method for imaging the interior of a bodily cavity of a patient; a system, device and method for mapping the interior of a bodily cavity of a patient; a method for implanting a nucleus pulposus replacement device, a delivery device for implanting a nucleus pulposus replacement device and a sealing device for sealing a bodily cavity of a patient.

### BACKGROUND ART

The human intervertebral disc (IVD) is a structure composed of a complex arrangement of various connective tissues. The structure of the IVD allows for its role in the effect of a functioning spinal column. Degeneration of the IVD is a consequence of aging and may begin as early as the first decade of life in males and the second decade in females. Disc degeneration plays a significant role in the aetiology of nucleus pulposus herniation, spinal stenosis and segmental, spinal stability. Furthermore, IVD degeneration is implicated as a causative factor in mechanical lower back pain.

Over the years, there have been several suggestions and techniques relating to the development of prosthetic IVD replacement devices. Such devices include replacement of the entire intervertebral disc, and replacement of the nucleus pulposus only. Other methods of treatment include therapies for degenerated discs such as fusion and discectomy. Artificial devices are intended to restore or preserve the natural biomechanics of the intervertebral segment and to reduce further degeneration of adjacent levels of the spine.

Devices to replace the entire intervertebral disc include mechanical fixation devices which preserve the intersegmental stability using metallic end plates affixed to adjacent vertebra and an elastomeric rubber “nucleus” between the end plates. Other type of devices include “metal on metal” prostheses extending across adjacent vertebra.

Nucleus pulposus replacement devices involve substitution or augmentation of the nucleus pulposus in the event of IVD degeneration with normal annular architecture. Such devices include a prosthetic disc nucleus (eg. The PDN™ of RayMedica Inc., Minneapolis, Minn.), consisting of hyaluronic acid (hydroscopic gel) within a semi-permeable membrane that is enclosed in a woven jacket. A pair of these devices is inserted per level of the spine and, with time, an increased water content of the devices from absorption results in the volume of the devices expanding. Another such nucleus pulposus replacement device, is the Aquarelle™ Hydrogel Disc Nucleus (Stryker Howmedica Osteonics, Rutherford, N.J.). This device consists of a hydrogel disc nucleus which is inserted, using instrumentation, into the intervertebral disc via a hole in the annulus, the hole having a cross-sectional area approximately one-quarter of that of the implant. The implant is composed of polyvinyl alcohol and water, its water content being high at intradiscal pressures found in the human lumbar spine. This property assists the implant to have a relatively low modulus of elasticity which allows it to conform to the vertebral end plates of the adjacent vertebra.

The present inventor has identified short comings within the prior art and has developed a system which seeks to alleviate some of the short failings.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this' specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention is a system for imaging the interior of a bodily cavity of a patient comprising:

a first imaging means positionable within and for producing a first image of said interior; and

at least a second imaging means positionable within and for producing a second image of said interior;

wherein said second imaging means is movable relative to the first imaging means and positionable in a location wherein said first image depicts the location of the second imaging means.

In an embodiment of the first aspect, the system further comprises a display means for displaying said first and second images. Preferably the display means comprises a first monitor for said first image and at least a second monitor for said at least second image. Alternatively the display means comprises one monitor that displays said first image and said at least second image. Preferably the system further comprising an illuminating means for illuminating said cavity.

In another embodiment of the first aspect, the system further comprises a tissue ablation means for ablating tissue in the bodily cavity, said ablation means being movable relative to the first imaging means. Preferably, the first image depicts the location and orientation of the tissue ablation means. More preferably, the tissue ablation means is located adjacent to the second imaging means and the second image depicts the tissue undergoing ablation.

In a further embodiment of the first aspect, the tissue ablation means is preferably a radio-frequency ablation device or a plasma discharge device.

In yet another embodiment of the first aspect, the first imaging means is a camera, and more preferably the camera is a video camera. Preferably the second imaging means is a camera, and more preferably the camera is a video camera. In each case, the camera can be an analogue or digital camera.

In yet a further embodiment of the first aspect, the second imaging means is an arthroscope. Preferably the arthroscope includes a flexible elongate portion having a camera positioned thereon that is adapted to be insertable into the cavity, the flexible elongate portion allowing the portion of the periphery of the bodily cavity adjacent to the point of entry of the arthroscope to be viewed and accessed. More preferably, the first imaging means and the second imaging means are positioned on a support member and maintained in a spaced apart relationship relative to each other. Preferably the support member is at least partially insertable within said bodily cavity. Preferably the first imaging means is an arthroscope.

In still another embodiment of the first aspect, the system further comprises a position indication means variably positionable within said bodily cavity;

a position detection means for receiving a signal from the position indication means; and

a processor means that analyses said signal and provides an output indicative of the location of the position indication means relative to the position detection means.

Preferably the signal is selected from the group comprising Infra-red radiation, ultrasonic radiation, magnetic radiation, radio-frequency radiation, X-ray radiation and an optical image signal.

Preferably the position indication means is a transmitter means and the position detection means is a receiver means. Alternatively, the position indication means is a reflector means and the position detection means is a transceiver means and further wherein said signal is firstly transmitted from said transceiver means and is then reflected by said reflector means back to said transceiver means.

Preferably, the output of the processor means is used to build a map of the bodily cavity. More preferably, the system further comprising a comparator display that displays a visual comparison of said map and a real image of said bodily cavity. Preferably, the comparator display allows determination of the orientation of the second imaging means in said cavity. Preferably, the transmitter means is positionable at or adjacent the location of said second imaging means.

Preferably, the real image is obtained using an imaging technique selected from the group comprising X-ray imaging, magnetic resonance imaging, and computer tomography imaging. Preferably, the real image is obtained prior to mapping of said bodily cavity. Alternatively, the real image is obtained during said mapping of said bodily cavity. Preferably, the real image is continuously updated during said mapping of said bodily cavity. Preferably, the receiver means is positionable outside said bodily cavity. Alternatively, the receiver means is positionable within said bodily cavity

In still yet another embodiment of the present aspect, the bodily cavity is the nuclear space of an intervertebral disc.

In still yet a further embodiment of the present aspect, the bodily cavity is a joint cavity.

In a second aspect, the present invention is a system for mapping the interior of a bodily cavity of a patient, the system comprising:

a position indication means variably positionable within said bodily cavity;

a position detection means for receiving a signal from the position indication means; and

a processor means that analyses said signal and provides an output indicative of the location of the position indication means relative to the position detection means.

In an embodiment of the second aspect, preferably the position indication means is a transmitter means and the position detection means is a receiver means. Alternatively, the position indication means is a reflector means and the position detection means is a transceiver means and further wherein said signal is firstly transmitted from said transceiver means and is then reflected by said reflector means back to said transceiver means.

Preferably, the signal is selected from the group comprising Infra-red radiation, ultrasonic radiation, magnetic radiation, radio-frequency radiation, X-ray radiation and an optical image signal.

In another embodiment of the second aspect, the output of the processor means is preferably used to build a map of the bodily cavity. Preferably, the system further comprises a comparator display that displays a visual comparison of said map and a real image of said bodily cavity. Preferably, the real image is obtained using an imaging technique selected from the group comprising X-ray imaging, magnetic resonance imaging, and computer tomography imaging. Preferably, the real image is obtained prior to mapping of said bodily cavity. Alternatively, the real image is obtained during said mapping of said bodily cavity.

In a further embodiment of the second aspect, the system further comprises a tissue ablation means for ablating tissue in the bodily cavity, said ablation means being movable relative to the position detection means and positioned adjacent to said position indication means such that the location of the position indication means is indicative of the location of the ablation means. Preferably, the tissue ablation means is a radio-frequency ablation device. Alternatively, the tissue ablation means is a plasma discharge device. Preferably, the real image is continuously updated during said mapping of said bodily cavity.

In yet another embodiment of the present aspect, the position detection means is positionable outside said bodily cavity. Alternatively, position detection means is positionable within said bodily cavity.

In yet a further embodiment of the second aspect, the system further comprises a viewing means for imaging the interior of a bodily cavity of a patient, said viewing means comprising:

a first imaging means positionable within and for producing a first image of said interior; and

at least a second imaging means positionable within and for producing a second image of said interior;

wherein said second imaging means is movable relative to the first imaging means and positionable in a location wherein said first image depicts the location of the second imaging means.

In yet a further embodiment of the second aspect, the bodily cavity is the nuclear space of an intervertebral disc.

In still another embodiment of the second aspect, the bodily cavity is a joint cavity.

In a third aspect, the present invention is a method of imaging the interior of a bodily cavity of a patient, the method comprising the steps of:

producing a first image of said interior wherein said first image is produced by a first imaging means positionable within said interior;

producing at least a second image of said interior wherein said at least a second image is produced by a second imaging means positionable within said interior; and

positioning said first imaging means in a location wherein said first image depicts the location of the second imaging means.

In an embodiment of the third aspect, the method includes the use of the system of the first aspect and associated embodiments.

In a fourth aspect, the present invention is a method of mapping the interior of a bodily cavity of a patient, the method comprising the steps of:

introducing a position indication means within the bodily cavity, said position indication means being variably positionable within said bodily cavity;

positioning a position detection means to receive a signal from the position indication means; and

analysing said signal and providing an output indicative of the location of the position indication means relative to a position detection means.

Preferably the signal is selected from the group comprising Infra-red radiation, ultrasonic radiation, magnetic radiation, radio-frequency radiation, X-ray radiation and an optical image signal.

In one embodiment of the fourth aspect, the analysing step can be performed by a processor means.

In another embodiment of the fourth aspect, the position indication means is a transmitter means and the position detection means is a receiver means. Alternatively, the position indication means is a reflector means and the position detection means is a transceiver means and further wherein said signal is firstly transmitted from said transceiver means and is then reflected by said reflector means back to said transceiver means.

In a further embodiment of the fourth aspect, the method further comprises a step of using said output to build a map of the bodily cavity. Preferably, the method further comprises a step of displaying said map of the bodily cavity on a display means. More preferably, the method further comprises a step of comparing said map with a real image of said bodily cavity.

Preferably, the real image is obtained using an imaging technique selected from the group comprising X-ray imaging, magnetic resonance imaging, and computer tomography imaging. Preferably, the step of comparing said map with said real image comprises the steps of:

determining the real position of said position detection means relative to the bodily cavity; and

superimposing said real position of said position detection means with said real image of said bodily cavity on said display means.

In yet another embodiment of the fourth aspect, the method further comprises the steps of:

ablating at least a portion of the bodily cavity using an ablation means; and

updating said map during said ablation.

In yet a further embodiment of the fourth aspect, the method includes the use of the system of the third aspect and associated embodiments.

In a fifth aspect, the present invention is a device for imaging the interior of a bodily cavity of a patient comprising:

a support member at least partially positionable within said interior;

a first imaging means engageable with said support member for producing a first image of said interior; and

at least a second imaging means engageable with said support member for producing a second image of said interior;

wherein said second imaging means is movable relative to the first imaging means and positionable at a location wherein said first image depicts the location of the second imaging means.

In an embodiment of the fifth aspect, the device further comprises a tissue ablation means for ablating tissue in said bodily cavity, said ablation means being engageable with said support member and being moveable relative to the first imaging means. Preferably, the tissue ablation means is located adjacent to the second imaging means and said first image depicts the location and orientation of the tissue ablation means.

In another embodiment of the fifth aspect, the device further includes at least some of the embodiments of the first aspect.

In a sixth aspect, the present invention is a device for mapping the interior of a bodily cavity of a patient, the device comprising:

a support member at least partially positionable within said bodily cavity;

a position indication means engageable with said support member and variably positionable within said bodily cavity;

a position detection means for receiving a signal from the position indication means; and

a processor means that analyses said signal and provides an output indicative of the location of the position indication means relative to the position detection means.

Preferably, the signal is selected from the group comprising Infra-red radiation, ultrasonic radiation, magnetic radiation, radio-frequency radiation, X-ray radiation and an optical image signal.

In an embodiment of the sixth aspect, the position detection means is engageable with said support member and positionable within said bodily cavity.

In another embodiment of the sixth aspect, the position indication means is a transmitter means and the position detection means is a receiver means. Alternatively, the position indication means is a reflector means and the position detection means is a transceiver means and further wherein said signal is firstly transmitted from said transceiver means and is then reflected by said reflector means back to said transceiver means.

In a further embodiment of the sixth aspect, the further comprising a tissue ablation means for ablating tissue in said bodily cavity, said ablation means being engageable with said support member and being moveable relative to the position detection means. Preferably the tissue ablation means is located adjacent to the position indication means.

In a seventh aspect, the present invention is a nucleus pulposus replacement device, the device comprising:

a non-constrained body of material introducible into and positionable within an annulus of an intervertebral disc of a patient;

wherein, following introduction into the annulus, the body of material undergoes a change from a first state to at least one second state such that when in said second state, the body of material is constrained within the annulus of the intervertebral disc.

In one embodiment of the seventh aspect, the body of material can substantially engage with and conform to the internal boundaries of the annulus of the intervertebral disc.

In another embodiment of the seventh aspect, the body of the device is made from a material having mechanical and visco-elastic properties suitable for structural support and load dampening in a spinal column of a patient.

In a still further embodiment of the seventh aspect, the device further comprises a non load-bearing membrane located at the periphery of the body of material, wherein the membrane is impermeable to the body of material.

Preferably, the material of the body is made of a silicone-based material. The material can be configured such that it changes from the first state to at least the second state cures after being implanted within the annulus of the intervertebral disc of the patient. In one embodiment, the body of material cures following implantation.

The device may be used to deliver bioactive substances to the annulus of the intervertebral disc of the patient. The bioactive substances may be substances which induce cell growth and/or cell reproduction.

In another embodiment of the seventh aspect, the device may be used as a drug delivery means for active and/or prophylactic treatment at the site of implantation.

In yet another embodiment of the seventh aspect, the device may include a radioactive substance and/or radiopaque marker for monitoring by X-ray postoperatively. Examples of such radiopaque marking and monitoring materials include barium sulphate and zinc oxide.

In an eighth aspect, the present invention is a method of replacing the nucleus pulposus of an intervertebral disc of a patient using the device of the first aspect, the method comprising the steps of:

(i) ablating the nuclear space of an intervertebral disc of a patient through an incision in an annulus of the intervertebral disc;

(ii) distracting the intervertebral disc;

(iii) introducing the body of material into the ablated nuclear space; and

(iv) allowing or causing the body of material to change from its first state to its said at least one second state such that it is constrained within the annulus of the intervertebral disc

In one embodiment of the eighth aspect, the incision through the annulus of the intervertebral disc is made through surgical approaches including a posterior-lateral approach, and/or an anterior approach, to the disc.

In another embodiment of the eighth aspect, the intervertebral disc can be distracted by way of an expansion means and/or by conventional traction. Preferably, the intervertebral disc is distracted by way of an expansion means passing through the incision in the annulus of the intervertebral disc and into the ablated nuclear space.

In a further embodiment of the eighth aspect, the expansion means used for step (ii) is a balloon device. The balloon device is preferably inflated by a fluid so as to distract the intervertebral disc. The fluid used to expand the balloon device is preferably biocompatible. Examples of suitable fluids include saline, PBS and sterile water. A further example includes the material of a nucleus pulposus replacement device according to the seventh aspect.

In another embodiment of the eighth aspect, the method can include a further step between steps (i) and (ii) wherein the nuclear space is irrigated so as to remove any debris, bone fragments and/or loose tissue.

In a further embodiment of the eighth aspect, the balloon expansion means can include radiopaque markers which allow the position of the balloon to be monitored by an imaging means, such as X-ray, and allow the pre-screening of disc placement.

In a still further embodiment of the eighth aspect, after the intervertebral disc has been distracted by the balloon expansion means, the balloon is preferably removed from the nuclear space. As a safety precaution, the nuclear space may then be injected with dilute barium sulphate-saline solution so as to determine if there is a leak into the spinal column.

In one embodiment of the eighth aspect, the body of material can be introduced into the nuclear space of an intervertebral disc of a patient using the delivery device as defined herein.

According to a ninth aspect, the present invention is the use of a silicone-based substance for the manufacture of a nucleus pulposus replacement device for the treatment of degenerative disc disease in the spine of a human being.

In this aspect, the nucleus pulposus replacement device can have one or more features according to the first aspect of the invention defined herein.

According to a tenth aspect, the present invention is a delivery device for implanting the device of the first aspect within the annulus of the intervertebral disc of a patient, the delivery device comprising:

a delivery device having a first end for the delivery of the body of material into the annulus whilst the material is in the first state; and

a disengagement means located at said first end of the delivery device;

wherein the disengagement means releases the delivery device from the body of material following delivery of the device into the annulus.

In one embodiment of the tenth aspect, the disengagement means is a crimping means for disengaging the delivery device from the body of material when the material has changed into said at least one second state.

In a further embodiment of the tenth aspect, the delivery device further comprises a flow restrictor which allows the body of material to readily pass through the delivery device and through the disengagement means but which inhibits the material from flowing in the opposite direction and back into the delivery device.

In a still further embodiment of the tenth aspect, the delivery device further comprises a non load-bearing expandable membrane. The membrane is preferably located adjacent the disengagement means and is positionable about the periphery of the body of material. The membrane is preferably impermeable to the body of material and remains about the body of material upon disengagement of the body of material from the delivery device by the disengagement means.

According to an eleventh aspect, the present invention is an intervertebral disc distraction device comprising:

an elongate delivery member; and

an expandable distraction member.

In this aspect, the expandable distraction device can be a balloon device that is expandable by a pressurised fluid. The balloon device is preferably inflated by a fluid so as to distract the intervertebral disc. The fluid used to expand the balloon device is preferably biocompatible. Examples of suitable fluids include saline, PBS and sterile water. Alternatively, the balloon device may also be expanded by a settable substance moveable from a first state to a second state wherein the settable substance is introduced into the balloon device while in a less viscous first state and moves to a second more viscous state after expansion of the balloon device. More preferably, the expandable distraction member comprises radiographic markers on its periphery for detection using radiographic techniques. Still more preferably, the expandable distraction member is formed from a radiographic material.

In an embodiment of the present aspect, the expansion member further comprises an introduction portion, wherein the introduction portion extends at least partially through the annulus of the intervertebral disc and wherein the fluid enters the balloon device through the introduction portion.

According to a twelfth aspect, the present invention is a sealing device for sealing a bodily cavity of a patient, the sealing device comprising:

an enclosed expandable membrane for insertion into said bodily cavity, said membrane comprising:
an internal surface;
an external surface; and
an aperture, said aperture providing a fluid pathway from the exterior of said membrane to the interior of said membrane;
wherein upon introduction of a fluid through said aperture and into the interior of the membrane, the membrane at least partially expands such that at least a portion of the external surface engages with at least a portion of the internal periphery of the bodily cavity; and
upon sealing of said aperture such that said fluid is retained with the interior of the membrane, the bodily cavity is sealed.

In an embodiment of the twelfth aspect, the membrane further comprises radiographic marking means such that the location of the membrane is monitorable using imaging techniques.

In another embodiment of the twelfth aspect, the fluid can move from a first state to a second state, wherein the second state has a viscosity greater than that of the first state.

In a further embodiment of the twelfth aspect, the aperture of the membrane is sealable by a sealing means wherein the sealing means is selected from the group comprising a valve, inherent properties of the material of the membrane, ultrasonic welding, temperature welding, UV light curing, sealant, clipping means and crimping

In yet a further embodiment of the twelfth aspect, the expandable membrane further comprises a introduction portion through which said fluid is introduced into interior of the membrane, the introduction portion being in fluid communication with said aperture. Preferably the introduction portion is formed integrally with said expandable membrane.

In yet another embodiment of the twelfth aspect, the expandable membrane is compressible such that the sealing device can be inserted into the bodily cavity through an access aperture extending from the exterior of the cavity to the interior of the cavity.

Preferably the expandable membrane further comprises a introduction portion through which said fluid is introduced into interior of the membrane, the introduction portion being in fluid communication with said aperture. Preferably the introduction portion is formed integrally with said expandable membrane. More preferably the introduction portion extends at least partially through the access aperture so as to provide a fluid pathway from the exterior of the bodily cavity to the interior of the membrane.

In still a further embodiment of the present aspect, the bodily cavity is the nuclear space of an intervertebral disc.

According to a thirteenth aspect, the present invention is a method of sealing a bodily cavity of a patient, the method comprising the steps of:

inserting an enclosed expandable membrane within the bodily cavity;

expanding the enclosed expandable membrane by introducing a fluid within the interior of the membrane through an aperture, said aperture extending from the exterior of said membrane to the interior of said membrane; and

sealing said membrane such that said fluid remains encapsulated within said membrane.

In an embodiment of the thirteenth aspect, the fluid can move from a first state to a second state, wherein the second state has a viscosity greater than that of the first state.

In another embodiment of the thirteenth aspect, the aperture of the membrane is sealed using a sealing means, wherein the sealing means is selected from the group comprising a valve, inherent properties of the material of the membrane, ultrasonic welding, temperature welding, UV light curing, sealant, clipping means and crimping.

In a further embodiment of the thirteenth aspect, the method includes the use of the sealing device of the twelfth aspect and associated embodiments.

### BRIEF DESCRIPTION OF THE DRAWINGS

By way of example only, preferred embodiments of the invention are now described with reference to the accompanying drawings, in which:

FIG. 1 shows a superior-transverse view through the intervertebral disc of a patient;

FIG. 2 shows an anterior view of a disco-vertebral joint of a patient;

FIG. 3 shows a sectional view of an annular device;

FIG. 4 shows a sectional view of a vertebral distraction device;

FIGS. 5(i) to 5(v) show steps in annulating and distracting the intervertebral disc of a patient;

FIGS. 6(i) to 6(iii) are superior-transverse views of implantation of a nucleus pulposus replacement device using a delivery device according to the present invention;

FIG. 7 depicts an example of a device for providing an interior map of the nuclear space of an intervertebral disc;

FIG. 8 depicts the use of the device of FIG. 7; and

FIG. 9 is a flow chart of a system of determining the geometry of the nuclear space of an intervertebral disc.

### DETAILED DESCRIPTION OF THE DRAWINGS

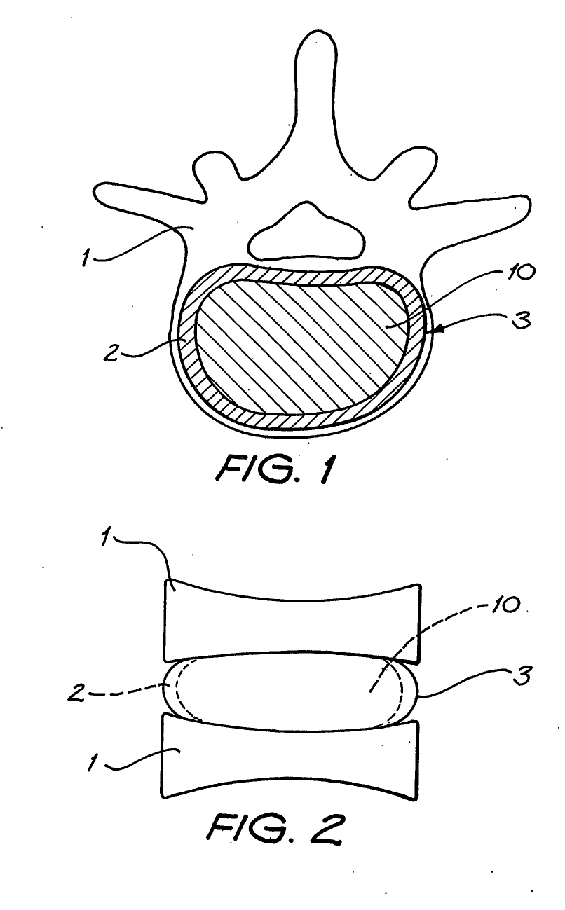

FIG. 1 depicts the annular wall 2 of an intervertebral disc 3 of a patient. A vertebra of the patient is depicted as item 1. The nucleus 10 of the intervertebral disc 3 is located within the annular wall 2.

FIG. 2 shows the intervertebral disc 3 of a patient located between two adjacent vertebrae 1 of the patient. The nucleus 10 of the inter vertebral disc 3 is bounded by the vertebrae 1 and the annular wall 2.

FIG. 3 is a sectional view of an annulatory device 20 for annulating the annular wall 2 of the intervertebral disc 3 of the patient. A localiser pin 21 is centrally positioned in the annulatory device 20. A trocar member 22 concentrically surrounds the localiser pin 21. An annulatory member 23 is located concentrically around the trocar member 22. The localising pin 21, trocar member 22 and the annulatory member 23 are in slidable engagement relative to each other.

In the depicted example of the annulatory device, the localiser pin 21 is formed of a biocompatible material, such as stainless steel and has a diameter of about 1.5 mm. The trocar member 22 has a distal end diameter of about 1.55 mm such that the localiser member 21 can slide within the trocar member 22. The outer diameter of the trocar member 22 is preferably about 3.5 mm. The distal end of the trocar member 22 preferably has a serrated edge such that it can engage fixedly with the outer surface of the annular wall of the intervertebral disc of a patient without dislodging therefrom. The annulatory member 23 also has a cutting edge at the distal end and has an outer diameter of about 4.5 mm. The inner diameter of the annulatory member 23 is slightly greater than the outer diameter of the trocar device such that sliding engagement is achieved.

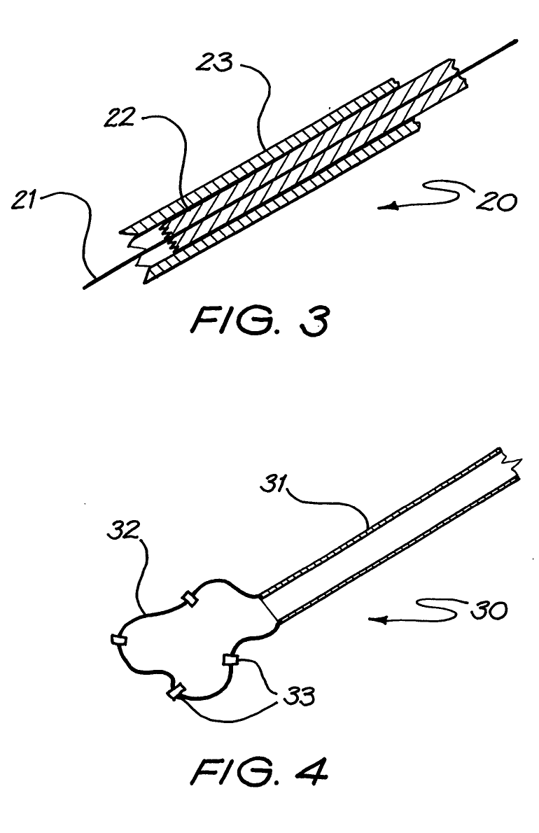

The distraction device 30, as shown in FIG. 4, can have an elongate delivery member 31 and an inflatable distraction member 32. Preferably, the inflatable distraction member 32 is an inflatable balloon device that is inflatable by a pressurised liquid. Preferably, the liquid used is a bio-inert material including saline and physiological fluid. Included on the periphery of the inflatable distraction member 32 are a plurality of radiopaque markers 33. The radiopaque markers 33 can be metallic or a metallic compound.

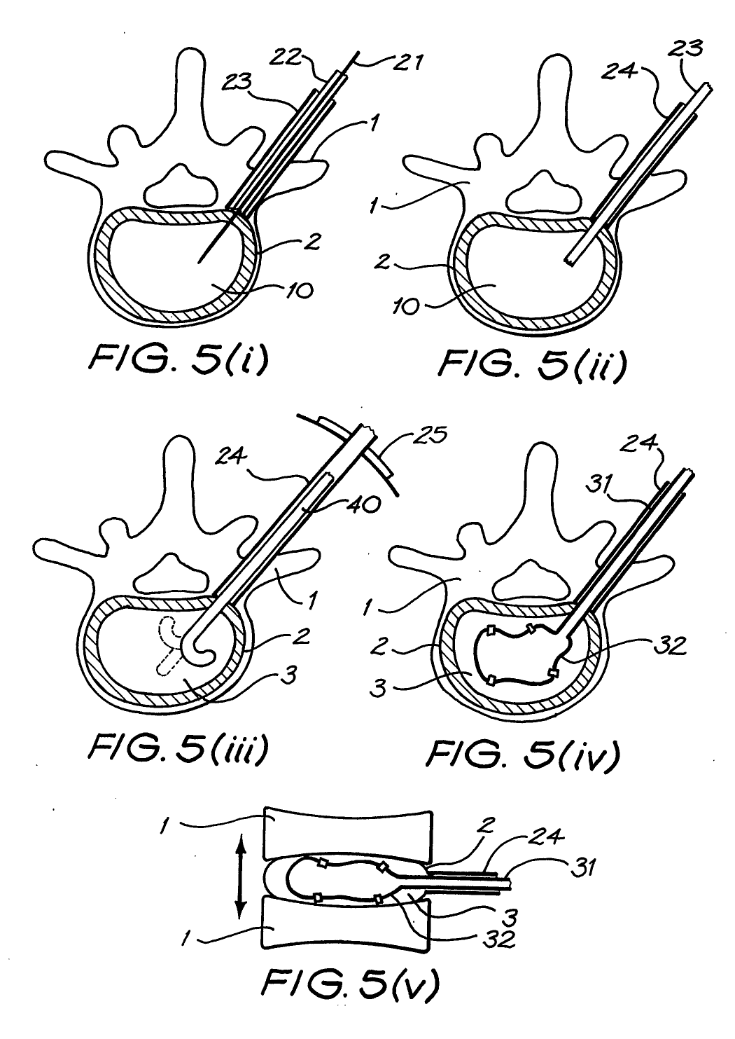

FIGS. 5(i) to 5(v) depict one example of the use of the annulatory device 20 of FIG. 3 and the use of the intervertebral disc distraction device. FIG. 5(i) depicts how the annulatory device 10 of FIG. 1 is engaged, in use, with the intervertebral disc 3 of a patient using a posterio-lateral surgical approach. Other approaches can be utilised. The trocar member 22 and the annulatory device 23 engage with the outer surface of the annular wall 2 of the intervertebral disc 3. The localiser pin 21 is initially used to locate the position at which the intervertebral disc 3 is to be annulated. Once the localiser pin 21 is in position and the annular wall 2 is perforated by the localiser pin 21, the trocar member 22 and the annulation member 23 are guided to the outer surface of the annular wall 2 using the localiser pin such that the trocar member 22 and the annulation member 23 are located as shown in FIG. 5(i).

The annulation member 23 is then used to annulate the annular wall 2 as shown in FIG. 5(ii). The cutting surface located at the distal end of the annulation member 23 allows cutting of the annular wall 2. The trocar member 22, by being engaged with the outer surface of the annular wall 2, provides support for the annulation member 23 whist the annular wall 2 is cut.

A working cannula 24 having an inner diameter slightly greater than the outer diameter of the annulation member 23 is then engaged with the outer surface of the annular wall 2 as shown in FIG. 5(ii). The annulation member 23 is used to guide the working cannula 24 into a position of engagement with the outer surface of the annular wall 2. The working cannula 24 can have engagement means for engaging with the outer surface of the annular wall 2. Such engagement means include pins, barbs or spikes. The localiser pin 21 and the trocar member 22 may be removed from the patient before or after the working cannula 24 is engaged with the outer surface of the annular wall 2. Once the working cannula 24 is engaged, the annulation device can be withdrawn from the patient through the working cannula 24. A stabilisation device 25 can be used external of the patient to stabilise the working cannula 24 (see FIG. 5(iii)).

An ablation device 40 is then inserted into the nucleus 10 of the intervertebral disc 3 through the working cannula as shown in FIG. 5(ii). The ablation device 40 is used to ablate the nucleus 10 of the intervertebral disc 3. The ablation device 40 can be for example a mechanical ablation device or a radio-frequency tissue ablation device. Once ablation is complete, the ablated nucleus 10 can be lavarged using saline or a physiological fluid. A radiopaque die for example dilute barium sulphate solution can be injected into the nucleus 10 and the patient scanned using radiographic techniques to determine the integrity of the annulus 22 and to determine if any leakage into the spinal canal of the patient has occurred. Arthroscopic techniques can also be employed through the working cannula 24 for inspection of the nucleus 10.

The intervertebral space between the vertebrae 1 of the patient is distracted following ablation of the nucleus 10. Distraction can be traction and/or internal distraction using the distraction device as shown in FIG. 4. The ablation device 40 is withdrawn from the patient through the working cannula 24 and the distraction member 32 of the distraction device is inserted into the ablated nucleus through the working cannula 24, with the delivery member 31 extending through the working cannula 24 and out of the patient as shown in FIG. 5(iv).

Pressurised fluid, for example saline solution, is injected through the delivery member 32 and into the distraction member 31, and pressurised for a period of time such the distraction of the vertebrae 1 adjacent the intervertebral disc 3 occurs. The patient can be imaged using radiographic techniques while the distraction member is expanded so as to determine the geometric parameters of the nucleus 10 of the intervertebral disc 3, as shown in FIG. 5(v).

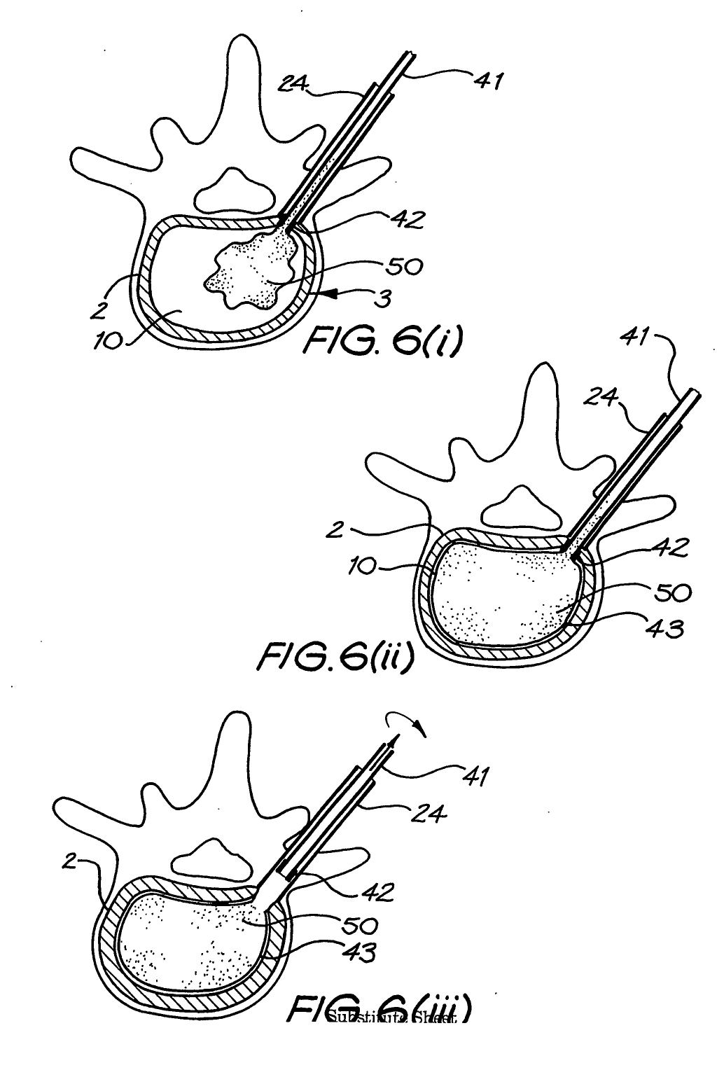

FIG. 6(i) depicts the implantation a nucleus pulposus replacement device according to the present invention within the nucleus 10 of the intervertebral disc 3 of a patient. Implantation of the device can follow the steps of the procedure as discussed with respect to FIG. 5.

A delivery device 41 is inserted within a working cannula 24, to the nuclear space 10 of the intervertebral disc 3. The material 50 from which the nucleus replacement device is formed is then injected through the delivery device 41 and into the nucleus 10 of the intervertebral disc 3, while the material 50 is in a first state suitable for injection. The material 50 can them conform substantially to the interior of the nucleus 10. The material 50 preferably has mechanical and visco-elastic properties suitable for pulposus replacement. An example of such a material 50 is a silicone-based material. Preferably, the material is self-curing by which the material changes to a second state having mechanical properties suitable for pulposus replacement.

Upon curing of the material 50, a disengagement means 42 of the delivery device 41 allows the delivery device 41 to be disengaged from the cured material 50 and withdrawn through the working cannula 24. Remaining within the nucleus 10 is the nucleus pulposus replacement device, substantially conforming to and constrained by the geometric boundaries of the nucleus, formed of the cured material 50.

FIG. 6(ii) shows a further example of implantation of the nuclear pulposus device, the device further comprising an outer membrane 43. During implantation, the membrane is fluidly attached to the delivery device 41 at the disengagement member. The delivery device 41 is inserted into the working cannula 24 such that the membrane 43 is located within the nucleus of the intervertebral disc 10. The material 50 from which the nucleus pulposus replacement device is formed from is delivered in the same manner as described in FIG. 6(i). Upon injection and at least a degree of pressurisation, the membrane 43 substantially conforms with the inner surface of the nucleus 10. Upon curing, the delivery device 41 is disengaged from the material 50 and the membrane 43 and removed from the working cannula 24.

FIG. 6(iii) shows an example of removal of the delivery device 41, following curing of the material 50. In this example, the delivery means is disengaged from the material 50 and the membrane by rotating the delivery device 40 within working cannula 24 and withdrawing the delivery device 41 through the working cannula 24.

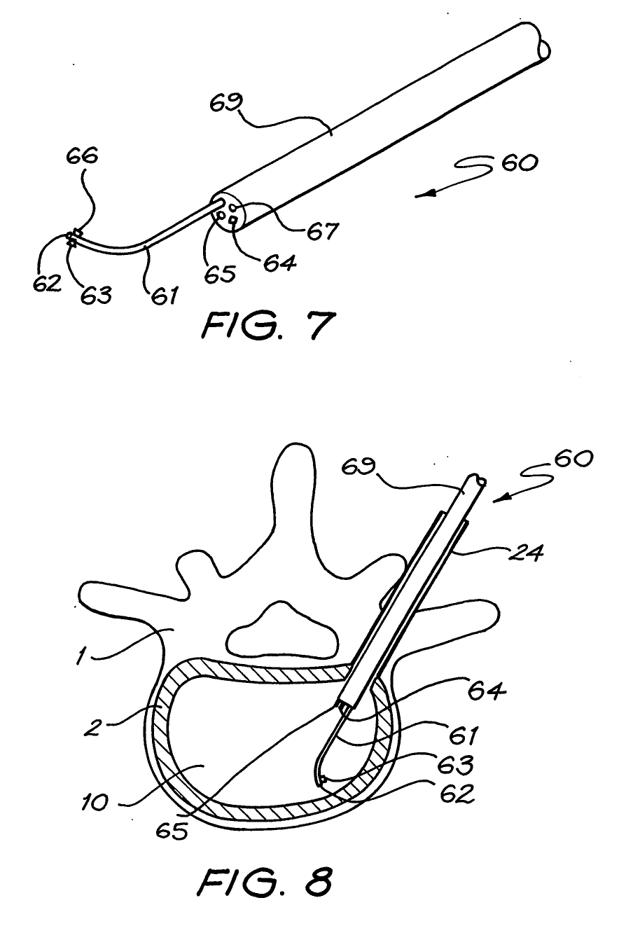

FIG. 7 depicts an example of a device 60 that can be used to generate an interior map of the nuclear space of an intervertebral disc 3 of a patient. The device 60 includes a transmitter 63 and a receiver 64. The transmitter 63 is located at the distal end of a flexible portion 61 of the device 60. The position and orientation of the flexible portion 61 is controllable by the surgeon from a position external the body of the patient, such that the position of the transmitter 63 is variable relative to the position of the receiver. The transmitter 63 transmits a signal to the receiver 64 that allows determination of the position of the transmitter relative to the receiver 64. An example of a suitable transmission mode is infra-red. In this example, the transmitter 63 is in direct line-of-sight from the receiver 64. Alternatively, a reflector means may be positioned at the distal end of the flexible portion and the transmitter located adjacent the receiver, or be integral with the receiver ain the form of a transceiver. The device 60 further includes an optical camera 62 located at the distal end of the flexible portion 61, and a second optical camera 65. A support member 69 maintains the optical camera 62 and the second optical camera 65 in a spaced apart relationship relative to each other such that an image provided by the second optical camera 65 depicts the location of the optical camera 62. The optical camera 62 and/or the second optical camera 65 may be a video camera. A digital image obtained by the second optical camera 65 can provide for position tracking of the optical camera 62 by image analysis techniques. The second optical camera 65 may be an arthroscope the flexible portion 61 may be a portion of the arthroscope. A light source 67 is also included as an illuminating means to allow imaging by the optical camera in the visible spectrum.

An ablation device 66 may also be also located at the distal end of the flexible portion 61. An example of a suitable ablation device includes a radio-frequency type probe another example of a suitable ablation device is a plasma discharge device.

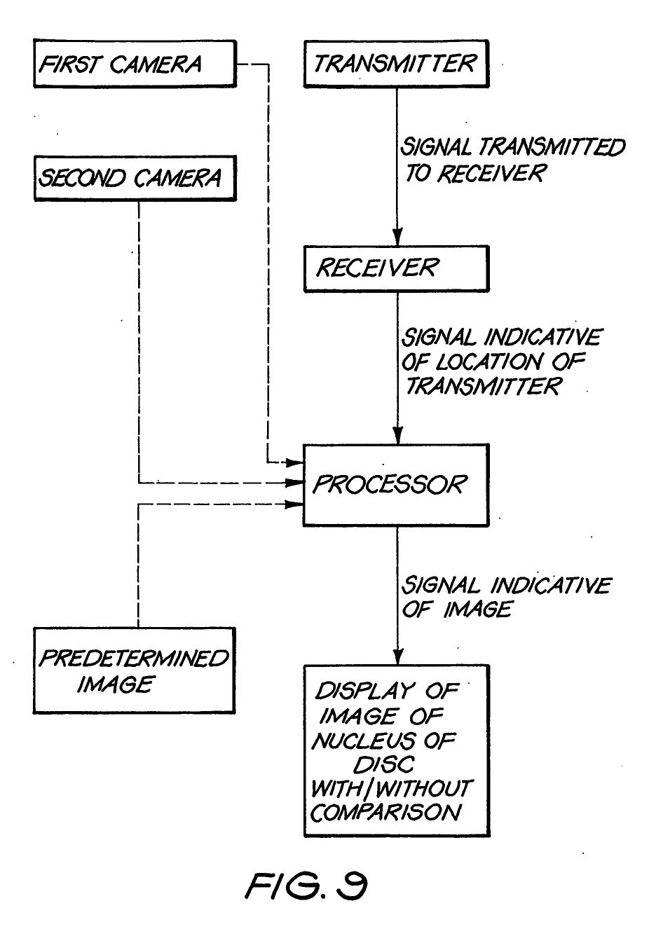

FIG. 8 depicts one example of the use of the device of FIG. 7. The device can be used for ablating the nucleus of the intervertebral disc of a patient and mapping the periphery of the nuclear space 10. The device 60 is at least partially inserted within the nuclear space 10 of the intervertebral disc of a patient through a working cannula 24. Examples of suitable surgical approaches include posterior-lateral approach and an anterior approach.

An optical camera 62 is located at the distal end of the flexible portion 61. The ablation device 66 is used to ablate the interior of the nuclear space 10. The region of the nuclear space at which ablation occurs can be imaged by the optical camera 62 and so provide an output visible to the surgeon during the procedure. The optical camera 65 allows for overall imaging of the distal end of the device 60 and the visual monitoring of the ablation device 66 during ablation assists in ensuring appropriate use of the ablation device 66 during the surgical procedure.

The transmitter 63, located at the distal end portion of the flexible portion 61 outputs a signal indicative of the location of the distal end of the device 60 relative to the receiver 64 and hence the location within the nucleus 10. In this example of the device, the receiver 64 is also located within the nucleus 10, although it will be appreciated that the receiver 64 could be located external of the body of the patient. Examples of a suitable modes of transmission the signal in the present example is infra-red transmission and radio-frequency transmission.

The position of the transmitter 63 relative to the receiver 64 can be processed by an external processor so as to allow generation of an internal map of the nucleus 10. Transmission of the signal from the transmitter 63 can be continuous, intermittent or user-operated. The user can position the distal end of the device 60 at a position within the nucleus 10, with the aid of the optical camera 65 and externally operate the transmitter 63 so as to determine the coordinates or position of the transmitter 63. Multiple transmissions at various locations along the periphery of the nucleus 10 allow development of a map or visual representation that is indicative of the volume and geometry of the nucleus 10.

The map or visual representation of the nucleus 10 output by the processor can be compared with real pre-obtained or simultaneously obtained images of the nucleus from various imaging techniques, such as X-ray, computer aided tomography, ultrasound and magnetic resonance imaging. Further to this, the image may be overlayed with the map of the nucleus to allow ready determination of the degree of ablation and/or monitoring of the position of the device 10.

FIG. 9 is a flow chart of representative of a system that uses the date of device 60. The system shown in FIG. 9 also provides visual monitoring of the ablation device 66 by the second optical camera 65, and visual monitoring of the portion of the nucleus being ablated and assessment of tissue by the optical camera 62. Visual monitoring can be provided by first monitor for display of an image from the optical camera 62 and a second monitor for display of an image from the second optical camera 65. Alternatively, a single monitor can display both the image from the optical camera 62 and the image from the second optical camera 65.

The image provided by the processor can be displayed on a comparator display with the internal map provided by the processor as described in reference to FIG. 8 with the real image in real time. As tissue is ablated by the ablation device, the map can be updated and compared with the real image. Such an updating of the image allows a user to determine the new real image of the cavity being mapped and allow a user to know where within the cavity that the ablation device is located, by way of superimposition of the updated image with the predetermined real image. The comparator display can be incorporated with the display which displays image from the optical camera 62 and the image from the second optical camera 65. It will be appreciated that the receiver may be located within or outside of the bodily cavity., and that any bodily cavity of a patient may be mapped in this way, including the interior nuclear space of an intervertebral disc of a patient.

A system incorporating such features enables a surgeon to assess the interior space of an intervertebral disc of a patient and to be provided with information as where a surgical instrument is located within the intervertebral disc. Furthermore, data indicative of the internal geometry of the intervertebral disc of a patient provided by such a system allows selection of an appropriately sized implant for nuclear pulposus replacement.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A system for imaging the interior of a bodily cavity of a patient comprising:
a first imaging means positionable within and for producing a first image of said interior; and
at least a second imaging means positionable within and for producing a second image of said interior;
wherein said second imaging means is movable relative to the first imaging means and positionable in a location wherein said first image depicts the location of the second imaging means.

2. The system according to claim 1 further comprising a display means for displaying said first and second images.

3. The system according to claim 1, further comprising a tissue ablation means for ablating tissue in the bodily cavity, said ablation means being movable relative to the first imaging means.

4. The system according to claim 3 wherein said first image depicts the location and orientation of the tissue ablation means.

5. The system according to claim 3, wherein the tissue ablation means is located adjacent to the second imaging means and said second image depicts the tissue undergoing ablation.

6. The system according to claim 3 wherein the tissue ablation means is a radio-frequency ablation device.

7. The system according to claim 3 wherein the tissue ablation means is a plasma discharge device.

8. The system according to claim 1 wherein the first imaging means is a camera.

9. The system according to claim 8 wherein the camera is a video camera.

10. The system according to claim 1 wherein the second imaging means is a camera.

11. The system according to claim 10 wherein the camera is a video camera.

12. The system according to claim 1 wherein the first imaging means is an arthroscope.

13. The system according to claim 1 wherein the second imaging means is an arthroscope.

14. The system according to claim 13 wherein the arthroscope includes a flexible elongate portion having a camera positioned thereon that is adapted to be insertable into the cavity.

15. The system according to claim 1 wherein the first imaging means and the second imaging means are positioned on a support member and maintained in a spaced apart relationship relative to each other.

16. The system according to claim 15, wherein the support member is at least partially insertable within said bodily cavity.

17. The system according to claim 13, further comprising:
a position indication means variably positionable within said bodily cavity;
a position detection means for receiving a signal from the position indication means; and
a processor means that analyses said signal and provides an output indicative of the location of the position indication means relative to the position detection means.

18. The system according to claim 17, wherein the position indication means is a transmitter means and the position detection means is a receiver means.

19. The system according to claim 17, wherein the position indication means is a reflector means and the position detection means is a transceiver means and further wherein said signal is firstly transmitted from said transceiver means and is then reflected by said reflector means back to said transceiver means.

20. The system according to claim 1 further comprising an illuminating means for illuminating said cavity.

21. The system according to claim 2 wherein said display means comprises a first monitor for said first image and at least a second monitor for said at least second image.

22. The system according to claim 2 wherein said display means comprises one monitor that displays said first image and said at least second image.

23. The system according to claim 17 wherein said output of the processor means is used to build a map of the bodily cavity.

24. The system according to claim 23 further comprising a comparator display that displays a visual comparison of said map and a real image of said bodily cavity.

25. The system according to claim 24 wherein the comparator display allows determination of the orientation of the second imaging means in said cavity.

26. The system according to claim 24 wherein said real image is obtained using an imaging technique selected from the group comprising X-ray imaging, magnetic resonance imaging, and computer tomography imaging.

27. The system according to claim 26 wherein said real image is obtained prior to mapping of said bodily cavity.

28. The system according to claim 26 wherein said real image is obtained during said mapping of said bodily cavity.

29. The system according to claim 28 wherein said real image is continuously updated during said mapping of said bodily cavity.

30. The system according to claim 18 wherein said transmitter means is positionable at or adjacent the location of said second imaging means.

31. The system according to claim 30 wherein said receiver means is positionable outside said bodily cavity.

32. The system according to claim 1 wherein said bodily cavity is the nuclear space of an intervertebral disc.

33. The system according to claim 1 wherein said bodily cavity is a joint cavity.

34. A system for mapping the interior of a bodily cavity of a patient, the system comprising:
a position indication means variably positionable within said bodily cavity;
a position detection means for receiving a signal from the position indication means; and
a processor means that analyses said signal and provides an output indicative of the location of the position indication means relative to the position detection means.

35. The system according to claim 34, wherein the position indication means is a transmitter means and the position detection means is a receiver means.

36. The system according to claim 34, wherein the position indication means is a reflector means and the position detection means is a transceiver means and further wherein said signal is firstly transmitted from said transceiver means and is then reflected by said reflector means back to said transceiver means.

37. The system according to claim 34 wherein said output of the processor means is used to build a map of the bodily cavity.

38. The system according to claim 37 further comprising a comparator display that displays a visual comparison of said map and a real image of said bodily cavity.

39. The system according to claim 38 wherein said real image is obtained using an imaging technique selected from the group comprising X-ray imaging, magnetic resonance imaging, and computer tomography imaging.

40. The system according to claim 38 wherein said real image is obtained prior to mapping of said bodily cavity.

41. The system according to claim 37, further comprising a tissue ablation means for ablating tissue in the bodily cavity, said ablation means being movable relative to the position detection means and positioned adjacent to said position indication means such that the location of the position indication means is indicative of the location of the ablation means.

42. The system according to claim 41 wherein the tissue ablation means is a radio-frequency ablation device.

43. The system according to claim 41 wherein the tissue ablation means is a plasma discharge device.

44. The system according to claim 38 wherein said real image is obtained during said mapping of said bodily cavity.

45. The system according to claim 38 wherein said real image is continuously updated during said mapping of said bodily cavity.

46. The system according to claim 34 wherein said position detection means is positionable outside said bodily cavity.

47. The system according to claim 34 wherein said position detection means is positionable within said bodily cavity.

48. The system according to claim 34, further comprising a viewing means for imaging the interior of a bodily cavity of a patient, said viewing means comprising:
a first imaging means positionable within and for producing a first image of said interior; and
at least a second imaging means positionable within and for producing a second image of said interior;
wherein said second imaging means is movable relative to the first imaging means and positionable in a location wherein said first image depicts the location of the second imaging means.

49. The system according to claim 34 wherein said bodily cavity is the nuclear space of an intervertebral disc.

50. The system according to claim 34 wherein said bodily cavity is a joint cavity.

51. A method of imaging the interior of a bodily cavity of a patient, the method comprising the steps of:
producing a first image of said interior wherein said first image is produced by a first imaging means positionable within said interior;
producing at least a second image of said interior wherein said at least a second image is produced by a second imaging means positionable within said interior; and
positioning said first imaging means in a location wherein said first image depicts the location of the second imaging means.

52. A method of mapping the interior of a bodily cavity of a patient, the method comprising the steps of:
introducing a position indication means within the bodily cavity, said position indication means being variably positionable within said bodily cavity;
positioning a position detection means to receive a signal from the position indication means; and
analysing said signal and providing an output indicative of the location of the position indication means relative to a position detection means.

53. The method of mapping according to claim 52, wherein the analysing step is performed by a processor means.

54. The method of mapping according to claim 52, wherein the position indication means is a transmitter means and the position detection means is a receiver means.

55. The method of mapping according to claim 52, wherein the position indication means is a reflector means and the position detection means is a transceiver means and further wherein said signal is firstly transmitted from said transceiver means and is then reflected by said reflector means back to said transceiver means.

56. The method of mapping according to claim 53, further comprising a step of using said output to build a map of the bodily cavity.

57. The method of mapping according to claim 56, further comprising a step of displaying said map of the bodily cavity on a display means.

58. The method of mapping according to claim 57, further comprising a step of comparing said map with a real image of said bodily cavity.

59. The method of mapping according to claim 58, wherein said real image is obtained using an imaging technique selected from the group comprising X-ray imaging, magnetic resonance imaging, and computer tomography imaging.

60. The method of mapping according to claim 58, wherein the step of comparing said map with said real image comprises the steps of:
determining the real position of said position detection means relative to the bodily cavity; and
superimposing said real position of said position detection means with said real image of said bodily cavity on said display means.

61. The method of mapping according to claim 56, further comprising the steps of:
ablating at least a portion of the bodily cavity using an ablation means; and
updating said map during said ablation.

62. A device for imaging the interior of a bodily cavity of a patient comprising:
a support member at least partially positionable within said interior;
a first imaging means engageable with said support member for producing a first image of said interior; and
at least a second imaging means engageable with said support member for producing a second image of said interior;
wherein said second imaging means is movable relative to the first imaging means and positionable at a location wherein said first image depicts the location of the second imaging means.

63. The device according to claim 62, further comprising a tissue ablation means for ablating tissue in said bodily cavity, said ablation means being engageable with said support member and being moveable relative to the first imaging means.

64. The device according to claim 63, wherein the tissue ablation means is located adjacent to the second imaging means and said first image depicts the location and orientation of the tissue ablation means.

65. A device for mapping the interior of a bodily cavity of a patient, the device comprising:
a support member at least partially positionable within said bodily cavity;
a position indication means engageable with said support member and variably positionable within said bodily cavity;
a position detection means for receiving a signal from the position indication means; and
a processor means that analyses said signal and provides an output indicative of the location of the position indication means relative to the position detection means.

66. The device according to claim 65, wherein said position detection means is engageable with said support member and positionable within said bodily cavity.

67. The device according to claim 65, wherein the position indication means is a transmitter means and the position detection means is a receiver means.

68. The device according to claim 65, wherein the position indication means is a reflector means and the position detection means is a transceiver means and further wherein said signal is firstly transmitted from said transceiver means and is then reflected by said reflector means back to said transceiver means.

69. The device according to claim 65, further comprising a tissue ablation means for ablating tissue in said bodily cavity, said ablation means being engageable with said support member and being moveable relative to the position detection means.

70. The device according to claim 69, wherein the tissue ablation means is located adjacent to the position indication means.
